(19) United States
(12) Patent Application Publication
(10) Pub. No.: US 2017/0184300 A1
(43) Pub. Date: Jun. 29, 2017

(54) ATOMIZING FRAGRANCE LAMP

(71) Applicant: DONGGUAN ESTIN HOME PRODUCTS CO., LTD, DongGuan City (CN)

(72) Inventor: Paul LIU, DongGuan City (CN)

(21) Appl. No.: 15/177,167

(22) Filed: Jun. 8, 2016

(30) Foreign Application Priority Data
Dec. 29, 2015 (CN) 201521132231.4

(51) Int. Cl. F21V 33/00; A61L 9/14; F21V 23/06; F21V 23/04; F21V 23/00

(52) U.S. Cl. CPC F21V 33/00; F21V 23/045; F21V 23/001; F21V 23/06; A61L 9/14; F21W 2131/30

(57) ABSTRACT

The present invention provides an atomizing fragrance lamp, comprising a shell, a circuit board, a light-emitting assembly, an atomizing assembly and a support base, wherein the shell comprises an upper chamber, a lower chamber and a top cover; two contact points connected with the circuit board extend from the bottom of the shell; the top cover is provided with a mist outlet; the atomizing assembly is mounted in the lower chamber together with the circuit board and electrically connected with the circuit board; and a color lamp of the light-emitting assembly is disposed in the upper chamber, extends out of the top cover through the mist outlet and is electrically connected with the circuit board. The atomizing fragrance lamp provided by the present invention is more compact in structure, attractive in appearance, small in size, convenient to carry, and meets the requirements of more people.

The current application claims a foreign priority to application number 201521132231.4 filed on Dec. 29, 2015 in China.

### BACKGROUND OF THE INVENTION

Technical Field

The present invention belongs to the technical field of household appliances,

and in particular to an atomizing fragrance lamp.

Description of Related Art

Fragrance help us to enjoy life and allows the air to be filled with aroma after

addition of essential oil, and after the essential oil is absorbed by a human body, fatigue can be alleviated, emotion can be regulated, and the conditions of some people suffering from chronic diseases can also be alleviated, thereby improving the quality of life. The fragrance appliances in the market are generally fragrance burners, in which the essential oil is heated to volatilize after being added into the burner, resulting in complicated and unsafe use. At present, there are also some atomizing fragrance devices, which however are more complex in structure and are generally featured with that an atomizing device and a light-emitting assembly are simultaneously disposed at the bottom of a shell, occupying a larger space and resulting in non-attractive appearance; and for people pursuing a quality life, these fragrance devices cannot meet their requirements and need to be improved.

### BRIEF SUMMARY OF THE INVENTION

To solve the problems above, the present invention provides an atomizing fragrance lamp, which is more compact in structure and more attractive in appearance, to solve the problem of complex structure in the prior art.

To achieve the effects as described above, the present invention employs a technical solution as follows: An atomizing fragrance lamp includes a shell, a circuit board, a light-emitting assembly, an atomizing assembly and a support base, where the shell includes an upper chamber, a lower chamber and a top cover; two contact points connected with the circuit board extend from the bottom of the shell; the top cover is provided with a mist outlet; the atomizing assembly is mounted in the lower chamber together with the circuit board and electrically connected with the circuit board; a color lamp of the light-emitting assembly is disposed in the upper chamber, extends out of the top cover through the mist outlet and is electrically connected with the circuit board; a chamber wall of the shell is provided with an atomizing opening which is engaged with the atomizing assembly; and two contact pieces which are not in mutual contact are disposed on parts on an upper end of the support base, corresponding to the contacts.

Preferably, the atomizing assembly includes a water storing box, a fan and an

atomizing piece, where the fan is disposed at the bottom of the lower chamber; and the fan and the atomizing piece are electrically connected with the circuit board to atomize water and deliver mist into the upper chamber to be discharged from the mist outlet.

Preferably, a chamber wall part of the shell extends upwards to form a cavity

bump with an opening at a top; the color lamp is disposed at a top of the bump and extends out of the top cover through the mist outlet; and a lamp line enters the lower chamber via a cavity to be electrically connected with the circuit board.

Preferably, the top cover recesses downwards to form the shell into a candle

shape, enhancing attractiveness at one hand and advantageously condensing the mist at the outlet and allowing the mist to flow back to the upper chamber for atomization again at the other hand.

Preferably, a base of the shell is provided with a plurality of support strips to

advantageously fix and support the circuit board and the water storing box; and the base part projects upwards to form a space for accommodating the fan for advantageously holding the fan.

Preferably, the support base is provided with a power plug to advantageously

allow the power line to pass through the support base to be connected with the contact pieces and in contact connection with the circuit board; and the bottom of the shell is provided with a switch having a timing function to facilitate artificial control and time regulation for setting a working state of the atomizing fragrance lamp.

Preferably, the atomizing fragrance lamp further includes a wireless receiving

portion, which is connected with the circuit board and used for receiving a control signal from external equipment in a wireless manner to control the light emitting of the light-emitting assembly, the working efficiency of the atomizing assembly and the on/off of the switch.

Preferably, the atomizing fragrance lamp further includes a casing, which includes a casing body and a base, where the support base is disposed on the base; the contact pieces are disposed on the bottom face inside the casing; and when in use, the shell is placed inside the casing body, with the contact points at the lower part of the shell being in contact with the contact pieces to realize wireless electric connection.

The present invention has the following advantageous effects: with the technical

solution provided by the present invention, an atomizing fragrance lamp with more compact structure and more attractive appearance can be obtained, and includes a shell, a circuit board, a light-emitting assembly, an atomizing assembly and a support base; the body part and support base of the atomizing fragrance lamp are electrified in a wireless contact manner; the atomizing assembly and the circuit board are mounted in the lower chamber; when in use, water and essential oil or perfume are added into the water storing box and then atomized through the atomizing piece, and diffuse through the fan, the mist enters the upper chamber and is discharged to the outside of the atomizing fragrance lamp from the mist outlet, meanwhile, the chamber wall extends and projects upwards to the upper chamber, and the color lamp of the light-emitting assembly extends out of the top cover; such a structure in which a cavity is designed to project in the upper chamber allows the atomizing fragrance lamp to be more compact and simpler in structure, small in occupied space and higher in a sense of layering; in addition, the mist flows out from the mist outlet and then is irradiated by the color lamp disposed at the mist outlet, forming a sense of misty beauty, and for another, the top cover is designed to be in a recess shape, allowing the whole structure of the atomizing fragrance lamp to take the shape of a candle, which is more attractive and also facilitates the flowing-back of water drops condensed from the mist; and the color of the color lamp is changeable, forming a cozy atmosphere more effectively.

### BRIEF DESCRIPTION OF THE SEVERAL VIEWS OF THE DRAWINGS

FIG. 1 is a schematic diagram of an inner planar structure of a first embodiment of the present invention.

FIG. 2 is a schematic diagram of a whole structure of the first embodiment.

FIG. 3 is a schematic diagram of a bottom structure of a shell of the first embodiment.

FIG. 4 is a schematic diagram of a structure of a support base of the first embodiment.

FIG. 5 is a schematic diagram of a stereoscopic structure inside the shell.

FIG. 6 is a schematic diagram of an inner structure of a second embodiment of the present invention.

FIG. 7 is a schematic diagram of a whole structure of the second embodiment.

FIG. 8 is a schematic diagram of a whole structure of the second embodiment from another direction.

FIG. 9 is a schematic diagram of an inner structure of the casing of the second embodiment.

### DETAILED DESCRIPTION OF THE INVENTION

The present invention will be further described below with reference to the drawings. It is understood that the description here is only for the purpose of illustrating specific implementation methods, and is not intended to limit the protection scope.

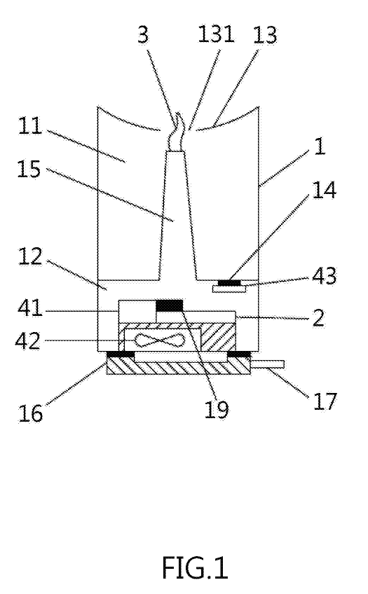

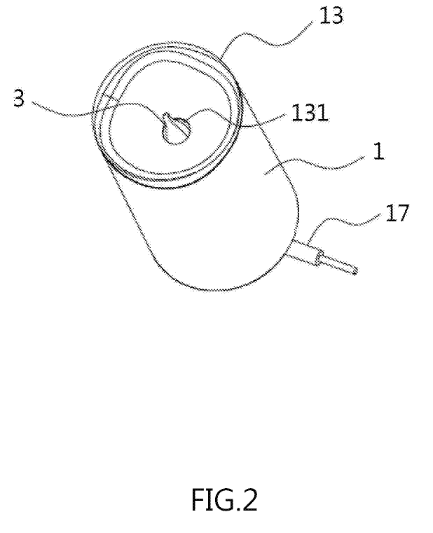

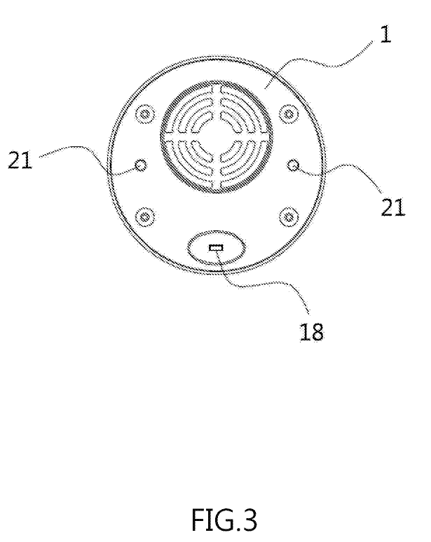

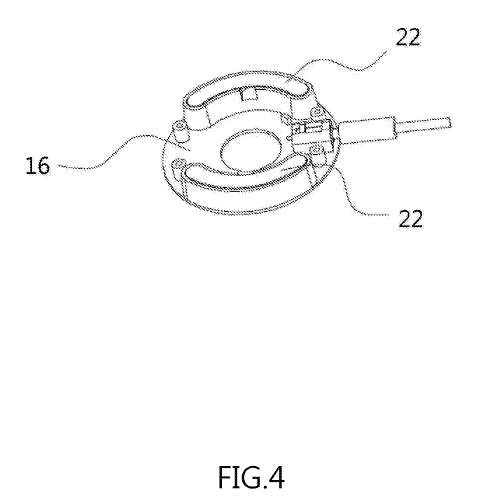

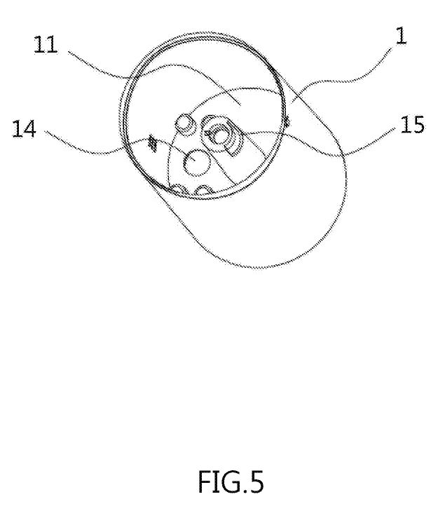

As shown in FIGS. 1 to 5 which illustrate the schematic diagrams of a first embodiment of the present invention, FIG. 1 is a schematic diagram of an inner structure of the first embodiment of the present invention; as shown in FIG. 1, an atomizing fragrance lamp includes a shell 1, a circuit board 2, a light-emitting assembly, an atomizing assembly and a support base 16, where the shell includes an upper chamber 11, a lower chamber 12 and a top cover 13; two contact points 21 connected with the circuit board 2 extend from the bottom of the shell 11; the top cover 13 is provided with a mist outlet 131; the atomizing assembly is mounted in the lower chamber 12 together with the circuit board 2 and electrically connected with the circuit board 2; a color lamp 3 of the light-emitting assembly is disposed in the upper chamber 11, extends out of the top cover 13 through the mist outlet 131 and is electrically connected with the circuit board 2; a chamber wall of the shell 1 is provided with an atomizing opening 14 which is engaged with the atomizing assembly; two contact pieces 22 which are not in mutual contact are disposed on parts on an upper end of the support base 16, corresponding to the contact points 21; the shell 1 can be freely taken up and put down from the support base 16, which is convenient and practical with a contact connection manner; after the atomizing assembly atomizes water drops in the lower chamber 12, water mist enters the upper chamber 11 and diffuses outwards from the mist outlet 131; in such a structure, the color lamp 3 of the light-emitting assembly and the mist outlet 131 are disposed together at first, and an extremely cozy and attractive feel is created with the mixture of a light effect when the mist is discharged; and moreover, compared with the structure in which both the light-emitting assembly and the atomizing assembly are disposed on the bottom of the shell 1, such a structure is more compact, has a sense of layering with a sequential arrangement and makes use of the space fully.

As a further improvement to the present embodiment, the atomizing assembly

includes a water storing box 41, a fan 42 and an atomizing piece 43, where the fan 42 is disposed at the bottom of the lower chamber 12; and the fan 42 and the atomizing piece 43 are electrically connected with the circuit board 2 to atomize water and deliver mist into the upper chamber 11 to be discharged from the mist outlet 131.

As a further improvement to the present embodiment, a chamber wall part of the

shell 1 extends upwards to form a cavity bump 15 with an opening at a top; the color lamp 3 is disposed at a top of the bump 15 and extends out of the top cover through the mist outlet 131; and a lamp line (not shown) enters the lower chamber 12 via a cavity to be electrically connected with the circuit board 2.

As a further improvement to the present embodiment, the top cover 13 recesses

downwards to form the shell 1 into a candle shape, enhancing attractiveness at one hand and advantageously condensing the mist at the outlet and allowing the mist to flow back to the upper chamber 11 for atomization again at the other hand.

As a further improvement to the present embodiment, a base of the shell 1 is

provided with a plurality of support strips to advantageously fix and support the circuit board 2 and the water storing box 41; and the base 15 part projects upwards to form a space for accommodating the fan for advantageously holding the fan 42.

As a further improvement to the present embodiment, the support base 16 is

provided with a power plug 17 to allow the power line to pass through the support base 16 to be connected with the contact pieces 22 and in contact connection with the circuit board 2; and the bottom of the shell is provided with a switch 18, which enables artificial control of the working state of the atomizing fragrance lamp and has a timing function, in such a way, a fixed time can be selected and regulated to enable the atomizing fragrance lamp to work at the fixed time.

As a further improvement to the present embodiment, the atomizing fragrance

lamp further includes a wireless receiving portion 19, which is connected with the circuit board 2 and used for receiving a control signal from external equipment in a wireless manner to control the light emitting of the light-emitting assembly, the working efficiency of the atomizing assembly and the on/off of the switch 18, which is convenient and free and is more flexible in operation.

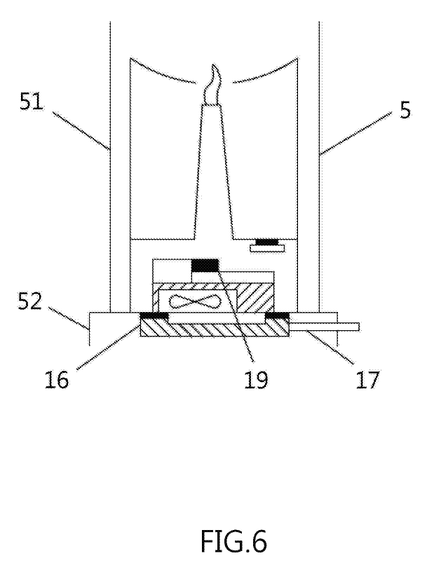

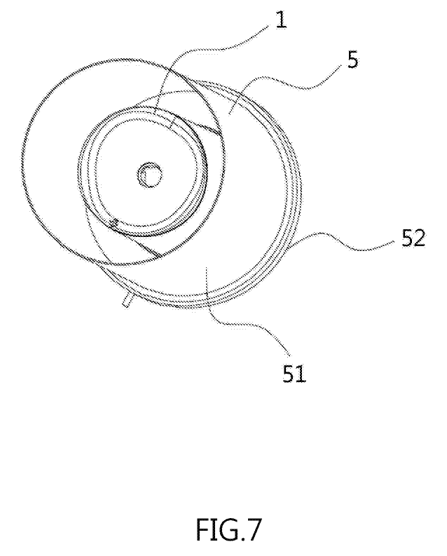

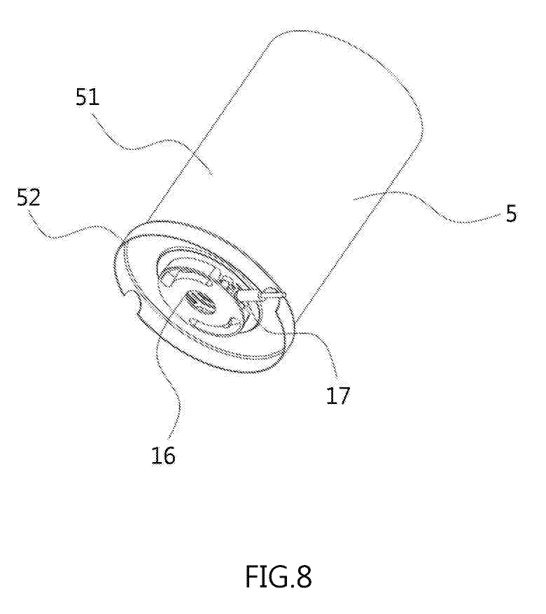

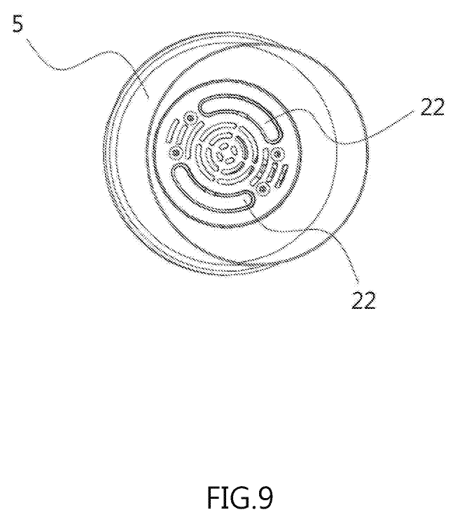

As shown in FIGS. 6 to 9 which illustrate schematic diagrams of a second embodiment of the present invention, the second embodiment is different from the first embodiment in that the atomizing fragrance lamp further includes a casing 5, which includes a casing body 51 and a base 52; the shell 1 is disposed inside the casing body 51 with a gap present therebetween; the support base 16 is disposed on the base 52; the contact pieces 22 are disposed on a bottom face inside the casing 5; when in use, the body part without the casing is placed in the casing 5, the contact points 21 come into contact with the contact pieces 22 to implement the manner of wireless electric connection; such an atomizing fragrance lamp having the casing is more stable after placement and more attractive in appearance; when in use, the body of the atomizing fragrance lamp is placed inside the casing 5 to form a whole structure like a candle holder; after the contact points 21 come into contact with the contact pieces 22 to turn on the switch 18 and switch on the power, the mist occasionally enters into the air to form a misty feel while the mist rises; and the casing 5 can be configured into different shapes and may be designed into a hollowed-out shape, which is more attractive.

With the technical solution as described above, the atomizing fragrance lamp

being more compact in structure and more attractive in shape and meeting the requirements of more people is obtained, which is small in size, convenient to carry and place; with the cavity bump disposed on the chamber wall, the color lamp is facilitated to extend out of the inner shell to emit light while the structure is simplified, thereby creating an extraordinary cozy atmosphere by the mist in the presence of the light; and moreover, when the body is placed on the support base, the power is also guaranteed to be on through angle rotation.

The embodiments of the present invention are merely intended to illustrate the

specific implementation manners thereof, but not intended to limit the protection scope of the present invention. Those skilled in the art may make some modifications inspired by the present embodiments. Therefore, any equivalent variations or modifications made according to the patent scope of the present invention are construed to fall under the patent scope of the Claims of the present invention.

What is claimed is:

1. An atomizing fragrance lamp, characterized by comprising a shell (1), a circuit board (2), a light-emitting assembly, an atomizing assembly and a support base (16), wherein the shell comprises an upper chamber (11), a lower chamber (12) and a top cover (13); two contact points (21) connected with the circuit board (2) extend from the bottom of the shell (1); the top cover (13) is provided with a mist outlet (131); the atomizing assembly is mounted in the lower chamber together with the circuit board (2) and electrically connected with the circuit board (2); a color lamp (3) of the light-emitting assembly is disposed in the upper chamber (11), extends out of the top cover (13) through the mist outlet (131) and is electrically connected with the circuit board (2); a chamber wall of the shell (1) is provided with an atomizing opening (14) which is engaged with the atomizing assembly (14); and two contact pieces (22) which are not in mutual contact are disposed on parts on an upper end of the support base (16), corresponding to the contact points (21).

2. The atomizing fragrance lamp according to claim 1, characterized in that the atomizing assembly comprises a water storing box (41), a fan (42) and an atomizing piece (43), wherein the fan (42) is disposed at the bottom of the lower chamber (12); and the fan (42) and the atomizing piece (43) are electrically connected with the circuit board (2) to atomize water and deliver mist into the upper chamber (11) to be discharged from the mist outlet (131).

3. The atomizing fragrance lamp according to claim 2, characterized in that a chamber wall part of the shell (1) extends upwards to form a cavity bump (15) with an opening at a top; the color lamp (3) is disposed at the top of the bump (15) and extends out of the top cover (13) through the mist outlet (131); and a lamp line enters the lower chamber (12) via a cavity to be electrically connected with the circuit board (2).

4. The atomizing fragrance lamp according to claim 3, characterized in that the top cover (13) recesses downwards to form the shell (1) into a candle shape, enhancing attractiveness at one hand and advantageously condensing the mist at the outlet and allowing the mist to flow back to the upper chamber (11) for atomization again at the other hand.

5. The atomizing fragrance lamp according to claim 4, characterized in that a base of the shell (1) is provided with a plurality of support strips to advantageously fix and support the circuit board (2) and the water storing box (41); and the base part projects upwards to form a space for accommodating the fan for advantageously holding the fan (42).

6. The atomizing fragrance lamp according to claim 5, characterized in that the support base (16) is provided with a power plug (17) to advantageously allow a power line to pass through the support base (16) to be connected with the contact pieces (22) and in contact connection with the contact points (21); and the bottom of the shell (1) is provided with a switch (18) having a timing function to facilitate artificial control and setting of a working state of the atomizing fragrance lamp.

7. The atomizing fragrance lamp according to claim 6, characterized by further comprising a wireless receiving portion (19), which is connected with the circuit board (2) and used for receiving a control signal from external equipment in a wireless manner to control the light emitting of the light-emitting assembly, the working efficiency of the atomizing assembly and the on/off of the switch (18).

8. The atomizing fragrance lamp according to claims 1, characterized by further comprising a casing (5), which comprises a casing body (51) and a base (52), wherein the support base (16) is disposed on the base; the contact pieces (22) are disposed on a bottom face inside the casing (5); and the shell (1) is placed inside the casing body (51).

9. The atomizing fragrance lamp according to claims 2, characterized by further comprising a casing (5), which comprises a casing body (51) and a base (52), wherein the support base (16) is disposed on the base; the contact pieces (22) are disposed on a bottom face inside the casing (5); and the shell (1) is placed inside the casing body (51).

10. The atomizing fragrance lamp according to claims 3, characterized by further comprising a casing (5), which comprises a casing body (51) and a base (52), wherein the support base (16) is disposed on the base; the contact pieces (22) are disposed on a bottom face inside the casing (5); and the shell (1) is placed inside the casing body (51).

11. The atomizing fragrance lamp according to claims 4, characterized by further comprising a casing (5), which comprises a casing body (51) and a base (52), wherein the support base (16) is disposed on the base; the contact pieces (22) are disposed on a bottom face inside the casing (5); and the shell (1) is placed inside the casing body (51).

12. The atomizing fragrance lamp according to claims 5, characterized by further comprising a casing (5), which comprises a casing body (51) and a base (52), wherein the support base (16) is disposed on the base; the contact pieces (22) are disposed on a bottom face inside the casing (5); and the shell (1) is placed inside the casing body (51).

13. The atomizing fragrance lamp according to claims 6, characterized by further comprising a casing (5), which comprises a casing body (51) and a base (52), wherein the support base (16) is disposed on the base; the contact pieces (22) are disposed on a bottom face inside the casing (5); and the shell (1) is placed inside the casing body (51).

14. The atomizing fragrance lamp according to claims 7, characterized by further comprising a casing (5), which comprises a casing body (51) and a base (52), wherein the support base (16) is disposed on the base; the contact pieces (22) are disposed on a bottom face inside the casing (5); and the shell (1) is placed inside the casing body (51).
